(19) United States
(12) Patent Application Publication
(10) Pub. No.: US 2024/0394875 A1
(43) Pub. Date: Nov. 28, 2024

(54) IMAGING BASED REPERFUSION THERAPY MONITORING DEVICES, SYSTEMS, AND METHODS

(71) Applicant: KONINKLIJKE PHILIPS N.V., EINDHOVEN (NL)

(72) Inventors: Arjen VAN DER HORST, TILBURG (NL); James David CEZO, COLORADO SPRINGS, CO (US)

(21) Appl. No.: 18/693,725

(22) PCT Filed: Sep. 5, 2022
(86) PCT No.: PCT/EP2022/074643

§ 371 Date: Mar. 20, 2024

Related U.S. Application Data
(60) Provisional application No. 63/246,946, filed on Sep. 22, 2021.

(51) Int. Cl. G06T 7/00; A61B 6/00; A61B 6/46; A61M 1/36

(52) U.S. Cl. CPC G06T 7/0012; A61B 6/461; A61B 6/481; A61M 1/3613; G06T 2207/10116; G06T 2207/30048; G06T 2207/30104

(57) ABSTRACT

A system includes a processor circuit that receives first external imaging data of a first area of a heart of a patient associated with a first blood vessel with a blockage. The processor circuit determines a first measurement representative of the blood flow through the first area. The processor circuit receives second external imaging data of a second area of the heart associated with a second blood vessel of the heart lacking the blockage. The processor circuit determines a second measurement representative of the blood flow through the second area. The processor circuit determines a progression of a reperfusion therapy associated with the first area. The processor circuit outputs a visual representation of the progression to a display. To determine the progression, the processor circuit determines a parameter representative of a relative blood flow between the first and second areas based on the first measurement and the second measurement.

### TECHNICAL FIELD

The present disclosure relates generally to monitoring and/or assessment of a progression of a reperfusion therapy, and, in particular, to monitoring the progression of the reperfusion therapy based on external imaging data. More specifically, external imaging data of a first area of a heart of a patient and external imaging data of a second area of the heart may be used to determine a progression of a reperfusion therapy targeting the first area.

### BACKGROUND

A percutaneous coronary intervention (PCI) may be utilized to treat a blockage (e.g., an occlusion, a lesion, a stenosis, and/or the like) within a blood vessel. The PCI may include a therapeutic procedure, such as administration of a drug, angioplasty, placement of a stent, and/or the like, that reduces a size of the blockage or opens (e.g., widens) the lumen of the affected blood vessel. To that end, PCI can restore blood flow through a blood vessel and to tissue that receives blood/oxygen via the blood vessel. Moreover, before a PCI therapy is delivered, the reduction in blood flow caused by a blockage within a blood vessel may cause tissue that receives blood/oxygen from the vessel to experience ischemia. Accordingly, PCI may restore or increase blood flow to tissue that experienced ischemia, which may restore the health of the tissue. However, even after a PCI therapy is delivered, blood/oxygen may not always suitably re-perfuse through tissue that has experienced ischemia. In particular, the increase and/or reintroduction of blood flow through the ischemic tissue may trigger an inflammatory response and/or oxidative damage, known as reperfusion injury, along with or in place of restoration of normal function of the tissue.

### SUMMARY

Disclosed herein is a system configured to evaluate (e.g., assess), display, and/or control (e.g., modify) a progress of a reperfusion therapy targeting an area of a patient's body, such as a portion of the myocardium of the heart of the patient. The system may include a processing system, which may include a processor circuit, that may determine the progression of the reperfusion therapy based on external imaging data. For instance, the processing system may determine a measurement representative of blood flow through the area targeted by the reperfusion therapy based on external imaging data of the area (e.g., contrast blush imaging). The processing system may further use external imaging data to determine a measurement representative of blood flow through a different area, such as blood flow through relatively healthy tissue, which may not be targeted by the reperfusion therapy. The processing system may then determine a relative level of blood perfusion in the targeted area with respect to the different area (e.g., the healthy area) based on a comparison (e.g., a ratio) between the measurements. Further, by relating this comparison and/or relative level of blood perfusion to a progress of the reperfusion therapy, the processing system may determine the progression of the reperfusion therapy. The relative level of blood perfusion may be related to the progression via thresholding, for example. The determined progression of the reperfusion therapy may be output to a display and/or may be used to adaptively control one or more components of the system. For instance, an intravascular reperfusion therapy device, a contrast infusion pump and/or an imaging device used to obtain the external imaging data, and/or the like may be controlled based on the determined progression. As an illustrative example, the intravascular reperfusion therapy device may be configured to deliver the reperfusion therapy by causing a venous obstruction. Continuing with this example, when a determined progression of the reperfusion therapy indicates that blood flow to the targeted area is improving or has reached a level of blood flow relatively close to the blood flow through the healthy area, the processing system may control the intravascular reperfusion therapy device to gradually decrease the venous obstruction or complete (e.g., terminate) the administration of the reperfusion therapy.

In an exemplary aspect, a system is provided. The system includes a processor circuit configured to: receive first external imaging data of a first area of a heart of a patient associated with a first blood vessel with a blockage, wherein the first external imaging data includes blood flow through the first area; determine a first measurement representative of the blood flow through the first area; receive second external imaging data of a different, second area of the heart associated with a different, second blood vessel of the heart lacking the blockage, wherein the second external imaging data includes blood flow through the second area; determine a second measurement representative of the blood flow through the second area; determine a progression of a reperfusion therapy associated with the first area; and output a visual representation of the progression of the reperfusion therapy to a display in communication with the processor circuit, wherein, to determine the progression of the reperfusion therapy, the processor circuit is configured to determine a parameter representative of a relative blood flow between the first area and the second area based on the first measurement and the second measurement.

In some aspects, the first external imaging data and the second external imaging data are obtained with contrast agent. In some aspects, the processor circuit is further configured to: control, based on the parameter, an infusion pump in communication with the processor circuit to deliver the contrast agent to the first area and the second area. In some aspects, the system further comprises the infusion pump. In some aspects, the visual representation of the progression of the reperfusion therapy comprises a visual representation of a derivative with respect to time of the parameter. In some aspects, to determine the parameter, the processor circuit is configured to: determine a ratio of the first measurement and the second measurement. In some aspects, the first measurement comprises at least one of a wash-in rate, a wash-out rate, an intensity of the first external imaging data, a brightness of the first external imaging data, or a contrast velocity. In some aspects, the processor circuit is further configured to: receive selection of the first area, wherein the processor circuit is further configured to determine the first measurement responsive to the selection of the first area. In some aspects, the processor circuit is further configured to: identify the first area based on one or more features of the first external imaging data, wherein the processor circuit is further configured to determine the first measurement responsive to the identification of the first area. In some aspects, the one or more features of the first external imaging data comprise a stent, intravascular reperfusion therapy device, or the blockage. In some aspects, the first external imaging data comprises an x-ray image of the first area. In some aspects, the first area comprises a first portion of a myocardium of the heart and the second area comprises a different, second portion of the myocardium. In some aspects, the first blood vessel comprises a first coronary artery and the second blood vessel comprises a second coronary artery. In some aspects, the processor circuit is further configured to: control delivery of the reperfusion therapy based on the parameter. In some aspects, to control the delivery of the reperfusion therapy, the processor circuit is configured to: instruct an intravascular reperfusion therapy device in communication with the processor circuit and positioned within a vessel of the patient to control the blood flow through the first area. In some aspects, the vessel comprises a coronary vein. In some aspects, the system further includes the intravascular reperfusion therapy device.

In an exemplary aspect, a system is provided. The system includes a processor circuit configured to: receive first x-ray imaging data of a first area of a heart of a patient, wherein the first x-ray imaging data comprises blood flow through the first area from a first blood vessel with a blockage, wherein the first area of the heart comprises at least one of a first portion of the myocardium or the first blood vessel, wherein the first blood vessel comprises a first coronary artery; determine, using the first x-ray imaging data, a first measurement representative of the blood flow through the first area based on a contrast agent within the first area; receive second x-ray imaging data of a different, second area of the heart, wherein the second x-ray imaging data comprises blood flow through the second area from a different, second blood vessel lacking the blockage, wherein the second area comprises at least one of a second portion of the myocardium or the second blood vessel, wherein the second blood vessel comprises a second coronary artery; determine, using the second x-ray imaging data, a second measurement representative of the blood flow through the second area based on the contrast agent within the second area; determine a progression of a reperfusion therapy associated with the first area; and output a visual representation of the progression of the reperfusion therapy to a display in communication with the processor circuit, wherein, to determine the progression of the reperfusion therapy, the processor circuit is configured to determine a parameter representative of a relative blood flow between the first area and the second area based on the first measurement and the second measurement.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic, schematic view of a system, in accordance with at least one embodiment of the present disclosure.

FIG. 2 is a schematic diagram of a processor circuit, in accordance with at least one embodiment of the present disclosure.

FIG. 3A is diagram of a human heart with an obstruction, in accordance with at least one embodiment of the present disclosure.

FIG. 3B is diagram of the human heart following a percutaneous coronary intervention (PCI), in accordance with at least one embodiment of the present disclosure.

FIG. 3C is diagram of the human heart following a reperfusion therapy, in accordance with at least one embodiment of the present disclosure.

FIG. 4 is a flow diagram of a method for evaluating a progression of a reperfusion therapy, in accordance with at least one embodiment of the present disclosure.

FIG. 5 is a schematic diagram of a portion of a heart, in accordance with at least one embodiment of the present disclosure.

FIG. 6 is a schematic diagram of a portion of a heart, in accordance with at least one embodiment of the present disclosure.

FIG. 7 is a schematic diagram of a portion of a heart, in accordance with at least one embodiment of the present disclosure.

FIG. 8 is a diagrammatic, schematic view of a screen display, in accordance with at least one embodiment of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. Additionally, while the description below may refer to blood vessels, it will be understood that the present disclosure is not limited to such applications. For example, the devices, systems, and methods described herein may be used in any body chamber or body lumen, including an esophagus, veins, arteries, intestines, ventricles, atria, or any other body lumen and/or chamber. For the sake of brevity, however, the numerous iterations of these combinations will not be described separately.

Aspects of the present disclosure can include features described in App. No. 63/246,904, filed Sep. 22, 2021 (Atty Dkt No. 2021PF00224/44755.2211PV01), App. No. 63/246,963, filed Sep. 22, 2021 (Atty Dkt No. 2021PF00228/44755.2212PV01), App. No. 63/246,919, filed Sep. 22, 2021 (Atty Dkt No. 2021PF00225/44755.2213PV01), and App. No. 63/246,929, filed Sep. 22, 2021 (Atty Dkt No. 2021PF00226/44755.2214PV01), the entireties of which are incorporated by reference herein.

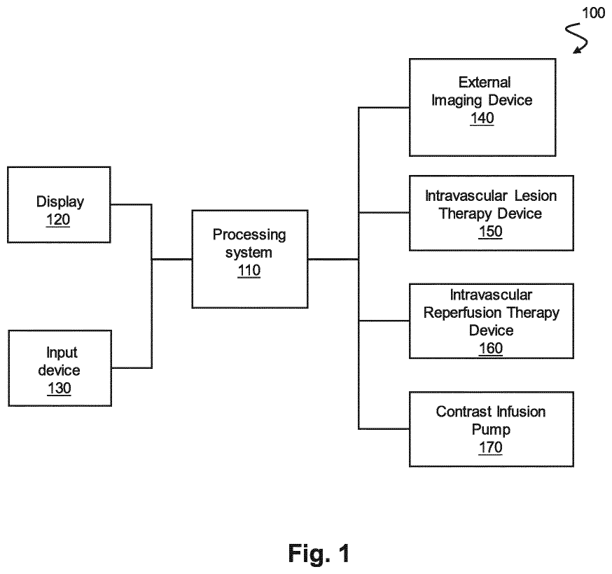

Referring to FIG. 1, shown therein is a system 100 according to an embodiment of the present disclosure. The system 100 can be configured to evaluate (e.g., assess), display, and/or control (e.g., modify) a progress of a reperfusion therapy targeting an area of a patient's body, such as a portion of the myocardium. For instance, the system 100 may be utilized to monitor and/or control reperfusion therapy such that injury to the myocardium following a percutaneous coronary intervention (PCI) is avoided or minimized, as described in greater detail below. In this regard, the system 100 may be used to assess coronary vessels and/or heart tissue (e.g., the myocardium) oxygenated by the coronary vessels. As illustrated, the system 100 includes a processing system 110 in communication with a display 120 (e.g., an electronic display), an input device 130 (e.g., a user input device), an external imaging device 140, an intravascular lesion therapy device 150 (e.g., intraluminal therapy device), an intravascular reperfusion therapy device 160 (e.g., intraluminal reperfusion therapy device), and a contrast infusion pump 170.

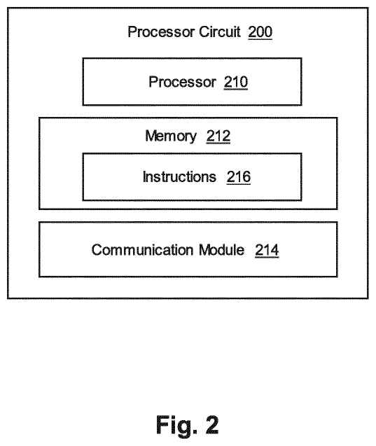

The processing system 110 is generally representative of any device suitable for performing the processing and analysis techniques disclosed herein. In some embodiments, the processing system 110 includes processor circuit, such as the processor circuit 200 of FIG. 2. In some embodiments, the processing system 110 is programmed to execute steps associated with the data acquisition, analysis, and/or instrument (e.g., device) control described herein. Accordingly, it is understood that any steps related to data acquisition, data processing, instrument control, and/or other processing or control aspects of the present disclosure may be implemented by the processor circuit (e.g., computing device) using corresponding instructions stored on or in a non-transitory computer readable medium accessible by the computing device. In some instances, the processing system 110 is a console device. Further, it is understood that in some instances the processing system 110 comprises one or a plurality of computing devices, such as computers, with one or a plurality of processor circuits. In that regard, it is particularly understood that the different processing and/or control aspects of the present disclosure may be implemented separately or within predefined groupings using a plurality of computing devices. Any divisions and/or combinations of the processing and/or control aspects described below across multiple computing devices are within the scope of the present disclosure.

FIG. 2 is a schematic diagram of a processor circuit 200, according to embodiments of the present disclosure. The processor circuit 200 may be implemented in the processing system 110 of FIG. 1. As shown, the processor circuit 200 may include a processor 210, a memory 212, and a communication module 214. These elements may be in direct or indirect communication with each other, for example via one or more buses.

The processor 210 may include a central processing unit (CPU), a digital signal processor (DSP), an ASIC, a controller, an FPGA, another hardware device, a firmware device, or any combination thereof configured to perform the operations described herein. The processor 210 may also be implemented as a combination of computing devices, e.g., a combination of a DSP and a microprocessor, a plurality of microprocessors, one or more microprocessors in conjunction with a DSP core, or any other such configuration.

The memory 212 may include a cache memory (e.g., a cache memory of the processor 210), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 212 includes a non-transitory computer-readable medium. The memory 212 may store instructions 216. The instructions 216 may include instructions that, when executed by the processor 210, cause the processor 210 to perform the operations described herein with reference to the processing system 110 (FIG. 1). Instructions 216 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 214 can include any electronic circuitry and/or logic circuitry to facilitate direct or indirect communication of data between various components of the processor circuit 200 and/or the processing system 110 (FIG. 1). Additionally or alternatively, the communication module 214 can facilitate communication of data between the processor circuit 200, the display 120 (e.g., a monitor), the input device 130, the external imaging device 140, the intravascular lesion therapy device 150, the intravascular reperfusion therapy device 160, the contrast infusion pump 170, and/or the like. In that regard, the communication module 214 can be an input/output (I/O) device interface, which may facilitate communicative coupling between the processor circuit 200 and (I/O) devices, such as the input device 130. Moreover, the communication module 214 may facilitate wireless and/or wired communication between various elements of the processor circuit 200 and/or the devices and systems of the system 100 using any suitable communication technology, such as a cable interface such as a USB, micro USB, Lightning, or FireWire interface, Bluetooth, Wi-Fi, ZigBee, Li-Fi, or cellular data connections such as 2G/GSM, 3G/UMTS, 4G/LTE/WiMax, or 5G.

Turning back now to FIG. 1, the external imaging device 140 can include an x-ray system, angiography system, fluoroscopy system, ultrasound system, computed tomography (CT) system, a magnetic resonance imaging (MRI) system, other suitable imaging devices, and/or combinations thereof. The external imaging device 140 may additionally or alternatively include a nuclear medicine imaging device, such as a gamma camera or a single-photon emission computed tomography (SPECT) system, other suitable devices, and/or combinations thereof. The external imaging device 140 can be configured to acquire imaging data of anatomy, such as the heart and blood vessels, while the external imaging device 140 is positioned outside of the body of the patient. The imaging data can be visualized in the form of two-dimensional and/or three-dimensional images of the heart, blood vessel, and/or other anatomy. In some embodiments, the imaging device 140 need not be an external device that is positioned outside the patient body. For example, the imaging device 140 can be an intracardiac echocardiography (ICE) catheter that obtains images while positioned within a heart chamber. In some embodiments, the imaging device 140 may be an external device in that is it is positioned outside of the particular anatomy that is being imaged (e.g., blood vessels and/or heart), but is positioned inside the patient body. For example, the imaging device 140 can be a transesophageal echocardiography (TEE) probe that obtains images while positioned within an esophagus.

Moreover, the external imaging device 140 may obtain images of the heart that are indicative of the health of the cardiac muscle or myocardium. In particular, the external imaging device 140 can be configured to acquire imaging data that illustrates myocardial perfusion (e.g., myocardial perfusion imaging (MPI) data). For example, MPI data can be collected by imaging a radiopharmaceutical agent, such as thallium, in the patient's heart muscle using a SPECT system. Additionally or alternatively, the imaging data may be obtained by imaging a contrast agent, which may be administered to the patient's vasculature manually or via the contrast infusion pump 170, for example. In any case, the imaging data can illustrate vasculature and/or muscle mass with blood flow and/or vasculature and/or muscle mass that lack of blood flow in areas of the heart.

The contrast infusion pump 170 may administer a contrast agent, which may alter an appearance (e.g., a brightness, an intensity, a contrast) of a feature within an external imaging data, such as the external imaging data obtained by the external imaging device 140. In that regard, the contrast infusion pump 170 may be configured to administer, to the patient, a contrast agent that is radiopaque and enhances the visibility of internal fluids or structures within a patient's anatomy. In some embodiments, for example, the contrast agent absorbs external x-rays from an x-ray source, resulting in decreased exposure on an x-ray detector in conjunction with the x-ray source. The contrast agent may be of any suitable material, chemical, or compound and, before administration to the patient, may be a liquid, powder, paste, tablet, or of any other suitable form. For example, the contrast agent may include iodine-based compounds, barium sulfate compounds, gadolinium-based compounds, microbubbles, or any other suitable compounds, which may be included in a solution or suspension, for example, for administration to the patient. In some embodiments, the contrast agent may include carbon dioxide, which may be a gas. In such cases, the contrast agent may decrease absorption of the external x-rays from the x-ray source, when administered. The contrast agent may additionally be referred to as a radiocontrast agent, a contrast dye, a radiocontrast dye, a contrast material, a radiocontrast material, a contrast media, or a radiocontrast media, among other terms. Further, in some embodiments, the contrast infusion pump 170 may be configured to combine or switch between different contrast agents, which may reduce stress on the patient's body. For instance, the contrast infusion pump 170 may administer a first contrast agent for a period of time and may subsequently administer a different, second contrast agent to the patient during an imaging procedure.

The intravascular lesion therapy device 150 may be any form of device, instrument, or probe sized and shaped to be positioned within a vessel. For example, the intravascular lesion therapy device 150 is generally representative of a guide wire, a catheter, or a guide catheter. However, in other embodiments, the intravascular lesion therapy device 150 may take other forms. In that regard, the intravascular lesion therapy device 150 may be a device configured to deliver a PCI therapy to a vessel. In particular, the intravascular lesion therapy device 150 may be an intravascular guidewire or catheter configured to ablate a lesion (e.g., a blockage) within the vessel, deploy a balloon, a stent, and/or drug to a target site within the vessel, and/or the like. That is, for example, the intravascular lesion therapy device 150 may be a stent or balloon delivery device (e.g., an angioplasty device), a thrombectomy device, an atherectomy device, and/or the like. In that regard, the intravascular lesion therapy device 150 may include a coil retriever, an aspiration (e.g., suction) device, and/or the like to assist in the removal of a clot or occlusion from the patient's vessel. In some embodiments, the intravascular lesion therapy device 150 may include a laser, a blade (e.g., knife), a sanding crown, and/or any suitable device that may assist in the cutting, shaving, sanding, vaporizing, and/or removal of atherosclerotic plaque from the patient's vessel. Additionally or alternatively, the intravascular lesion therapy device 150 may be the therapy itself delivered to the vessel. More specifically, the intravascular lesion therapy device 150 may represent a stent or balloon deployed to the vessel, a drug administered intra or extravascularly (e.g., orally), and/or the like. To that end, while the intravascular lesion therapy device 150 is illustrated as being communicatively coupled to the processing system 110, embodiments are not limited thereto.

In some embodiments, the intravascular reperfusion therapy device 160 may be a device, instrument, or probe sized and shaped to be positioned within a vessel. In particular, the intravascular reperfusion therapy device 160 may be a device or instrument configured to control reperfusion of blood flow into a target tissue area (e.g., capillary bed), such as a portion of the myocardium of a patient. In some embodiments, the target tissue area may be an ischemic area and/or an area of tissue that receives reduced blood flow due to a blockage in an associated vessel (e.g., an upstream artery). As described in greater detail below, treatment (e.g., therapy) directed to the vessel associated with the blockage, such as treatment via the intravascular lesion therapy device 150, may reintroduce or increase blood flow to the target tissue area. To reduce or prevent injury to the target tissue area resulting from this increased blood flow, the intravascular reperfusion therapy device 160 may be positioned intravascularly, such as within a coronary blood vessel, and may be configured to regulate blood flow to the target tissue. In some embodiments, the reperfusion therapy can include administration of anti-inflammatory drug(s) or nitric oxide (NO) to the patient. In some embodiments, the reperfusion therapy can include cold fluid that is provided via the arterial side.

In some embodiments, one or more of the external imaging device 140, the intravascular lesion therapy device 150, the intravascular reperfusion therapy device 160, and/or the contrast infusion pump 170, are located proximate one or more of the processing system 110, the display device 120, and/or the input device 130, such as in the same procedure room. In some embodiments, one or more of the external imaging device 140, the intravascular lesion therapy device 150, the intravascular reperfusion therapy device 160, and/or the contrast infusion pump 170 are located spaced from one or more of the processing system 110, the display device 120, and/or the input device 130, such as in different procedure rooms or facilities. For example, the external imaging device 140, the intravascular lesion therapy device 150, the intravascular reperfusion therapy device 160, and/or the contrast infusion pump 170 can be part of different systems that are communicatively coupled. In that regard, the processing system 110 can be configured to acquire the data collected from the components spaced therefrom and process the data as described herein. The external imaging device 140, the intravascular lesion therapy device 150, the intravascular reperfusion therapy device 160, and/or the contrast infusion pump 170 can be configured to transmit the collected data to the processing system 110.

The system 100 includes a display device 120 that is communicatively coupled to the processing system 110. In some embodiments, the display device 120 is a component of the processing system 110, while in other embodiments, the display device 120 is distinct from the processing system 110. In some embodiments, the display device 120 is a monitor integrated in a console device or a standalone monitor (e.g., a flat panel or flat screen monitor). The processing system 110 can be configured to generate a visual display (e.g., screen display) based on imaging data from the external imaging device 140. The processing system 110 can provide (e.g., output) the screen display to the display device 120. To that end, the display device 120 may be configured to output (e.g., display) a two-dimensional image and/or a two-dimensional representation of the heart, blood vessels, and/or other anatomy, which may be included in the screen display. In some embodiments, the display device 120 is configured to output a three-dimensional graphical representation of the heart, blood vessels, and/or other anatomy. For instance, the display device 120 may be a holographic display device configured to output a three-dimensional holographic display of anatomy. Any suitable display device is within the scope of this disclosure, including self-contained monitors, projection/screen systems, head-up display systems, etc. The display device can implement principles based on moving reflective microelectromechanical systems (MEMS), laser plasma, electro-holography, etc. In some embodiments, the display device 120 is implemented as a bedside controller having a touch-screen display as described, for example, in U.S. Provisional Application No. 62/049,265, titled “Bedside Controller for Assessment of Vessels and Associated Devices, Systems, and Methods,” and filed Sep. 11, 2014, the entirety of which is hereby incorporated by reference herein.

The system 100 includes an input device 130 that is communicatively coupled to the processing system 110. The input device 130 may be a peripheral device, such as a touch sensitive pad, a touch-screen, a joy-stick, a keyboard, mouse, trackball, a microphone, an imaging device, and/or the like. In other embodiments, the user interface device is part of the display device 120, which may be a touch-screen display, for example. Moreover, a user may provide an input to the processing system 110 via the input device 130. In particular, the input device 130 may enable a user to control, via inputs to the processing system 110, one or more of the components of the system 100, such as the external imaging device 140, the intravascular lesion therapy device 150, the intravascular reperfusion therapy device 160, the contrast infusion pump 170, or the processing system 110 itself. Additionally or alternatively, the input device 130 may facilitate interaction with a screen display provided at the display device 120. For instance, a user may select, edit, view, or interact with portions of the screen display (e.g., a GUI) provided at the display device 120 via the input device 130.

The system 100 can include various connectors, cables, interfaces, connections, etc., to communicate between the elements of the intravascular lesion therapy device 150, the intravascular reperfusion therapy device 160, the processing system 110, the external imaging device 140, the display device 120, and/or the input device 130. In some embodiments, for example, the communication module 214 (FIG. 2), which may be included in the processing system 110, may include such connectors, interfaces, and/or the like. In that regard, the processing system 110 can communicate and/or control one or more components of the system 110 via mechanical and/or electromechanical signaling and/or controls. Further, the illustrated communication pathways are exemplary in nature and should not be considered limiting in any way. In that regard, it is understood that any communication pathway between the components of system 100 may be utilized, including physical connections (including electrical, optical, and/or fluid connections), wireless connections, and/or combinations thereof. In that regard, it is understood that the one or more of the components of the system 100 can communicate via a wireless connection in some instances. In some instances, the one or more components of the system 100 and/or other systems (e.g., of a hospital or health services provider) communicate via a communication link over a network (e.g., intranet, internet, telecommunications network, and/or other network).

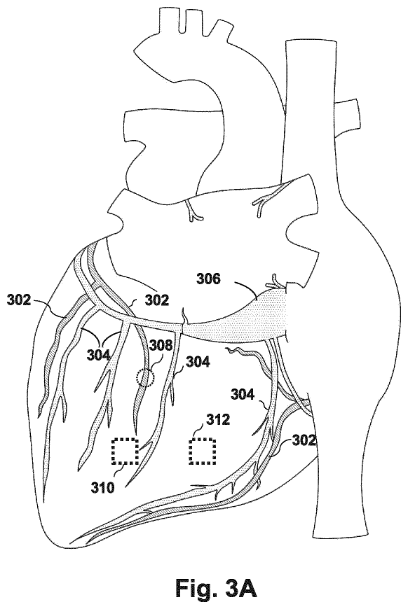

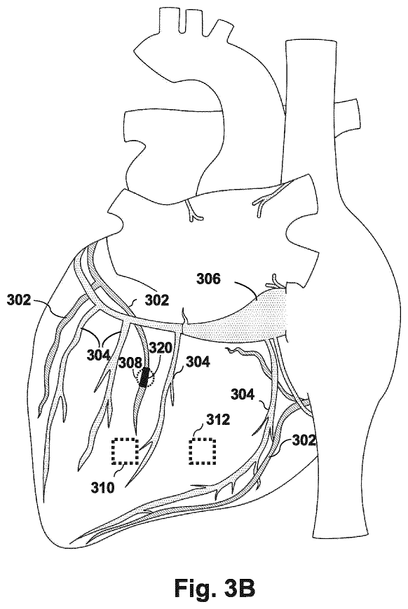

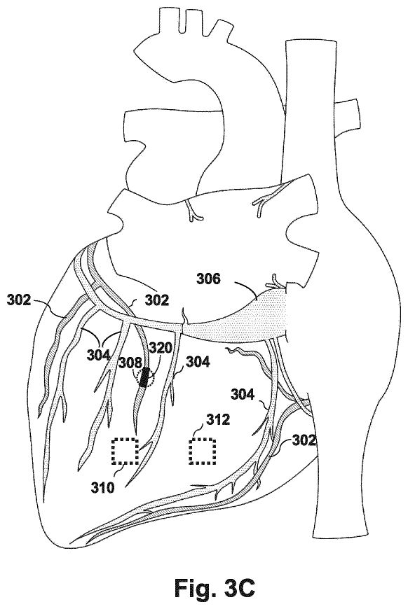

FIGS. 3A-3C illustrate a diagram of a human heart 300. As illustrated, the heart 300 includes coronary arteries 302 (illustrated with a first fill pattern) that deliver oxygenated blood to tissue, such as muscle tissue (e.g., myocardium), of the heart 300. The heart 300 further includes coronary veins 304 (illustrated with a second fill pattern), including a coronary sinus 306, that carry deoxygenated blood away from the tissue of the heart and towards a chamber (e.g., an atrium) of the heart 300.

In the diagram illustrated in FIG. 3A, a coronary artery 302 of the heart 300 includes a blockage 308 (e.g., an occlusion, a lesion, a stenosis, and/or the like). The blockage 308 may disrupt flow through the coronary artery 302. In particular, the blockage 308 may decrease the diameter of a portion of the lumen of the coronary artery 302, which may decrease the flow of blood through the portion of the lumen. As a result, a first area of tissue 310 (e.g., a portion of the myocardium) that is associated with (e.g., receives blood from) the coronary artery 302 with the blockage 308 may not receive a healthy amount of blood/oxygen. For instance, the blood/oxygen delivered to the first area of tissue 310 may not be sufficient to perfuse through (e.g., to be distributed across) the entire first area of tissue 310 in some cases. In this regard, the first area of tissue 310 may experience ischemia (e.g., a reduction in delivered blood/oxygen illustrated by a fill pattern), which may damage the first area of tissue 310. The illustrated different, second area of tissue 312 (e.g., a portion of the myocardium) may receive blood/oxygen from a different coronary artery 302 than the first area of tissue 310. In this regard, the second area of tissue 312 may remain relatively unaffected by the blockage 308. To that end, the second area of tissue 312 may receive a healthy amount of blood/oxygen, and the second area of tissue 312 may not experience ischemia. Accordingly, the second area of tissue 312 is illustrated as being healthy by a lack of the fill pattern shown in the first area of tissue 310.

In some embodiments, a percutaneous coronary intervention (PCI) may be utilized to treat the blockage 308. In particular, the PCI may include a therapeutic procedure that reduces a size of the blockage 308, opens (e.g., widens) the lumen of a vessel, and/or the like to restore blood flow through the vessel (e.g., the coronary artery 302) with the blockage 302. In that regard, the PCI may include, for example, angioplasty (e.g., deploying a balloon) and positioning a stent across the stenosis to open the vessel (e.g., the coronary artery 302 with the blockage). The PCI may additionally or alternatively include thrombectomy, atherectomy, administration of a drug and/or the like. To that end, the intravascular lesion therapy device 150 (FIG. 1) may facilitate and/or provide the PCI to a vessel having a blockage (e.g., blockage 308).

FIG. 3B illustrates a diagram of the heart 300 after delivery of a therapeutic procedure (e.g., post-treatment), such as PCI. In particular, FIG. 3B illustrates a stent 320 positioned within the coronary vessel at the site of the blockage 308. However, embodiments are not limited thereto. In that regard, the PCI delivered to the coronary artery 302 or a vessel with a blockage may include any suitable combination of the therapies described above.

As described above, the stent 320 and/or another suitable PCI (e.g., therapeutic procedure) may be provided to a vessel so that an effect of a blockage on blood flow through the vessel is reduced. In this regard, the placement of the stent 320 within the coronary artery 302 (e.g., at the site of the blockage 308) may open (e.g., widen) the portion of the lumen of the coronary artery 302 with the blockage 308, which may increase blood flow through the portion lumen. Moreover, the placement of the stent 320 within the heart 300 may increase blood flow downstream of the blockage 308, such as within vasculature that receives blood flow from the portion of the lumen. In this way, the vasculature (e.g., a capillary bed) that delivers blood/oxygen to the first area of tissue 310 may receive increased blood flow, which may increase blood/oxygen delivery to the first area of the tissue 310. To that end, blood/oxygen may re-perfuse the first area of the tissue 310. Accordingly, the stent 320 may reverse or reduce the ischemia experienced by the first area of the tissue 310. In this regard, the first area of the tissue 310 is illustrated in FIG. 3B with a different fill pattern than the fill pattern illustrated in FIG. 3A to demonstrate the increased blood/oxygen supplied to the first area of the tissue 310.

In some cases, blood/oxygen may not suitably perfuse through tissue associated with an occluded vessel (e.g., a vessel with a blockage), such as the first area of tissue 310, after delivery of a PCI therapy. For example, in some cases, the introduction and/or increase of blood flow to tissue that has experienced ischemia may result in reperfusion injury (e.g., ischemia-reperfusion injury). In particular, the returned blood flow may trigger an inflammatory response and/or oxidative damage along with or in place of restoration of normal function of the tissue. Inflammation, damage resulting from inflammation, and/or the oxidative damage may obstruct the flow of blood/oxygen within the tissue (e.g., within a capillary bed associated with the tissue). Accordingly, blood/oxygen may not be distributed throughout (e.g., perfuse through) the tissue at a healthy level even after the delivery of a PCI therapy. For instance, blood may preferentially flow through a first portion of the tissue lacking inflammation and/or damage and may flow through a second portion of the tissue with inflammation and/or damage to a lesser degree. As a result, the second portion of the tissue may continue to receive blood flow below a healthy level. In this regard, the first area of the tissue 310 is illustrated in FIG. 3B with a different fill pattern than the second area of the tissue 312 (e.g., a healthy area of tissue) to demonstrate that the stent 320 alone may not fully restore the health and/or functioning of the first area of the tissue 310.

Turning now to FIG. 3C, in some cases, the reperfusion of blood/oxygen within ischemic tissue may be further assisted and/or controlled by reperfusion therapy. To that end, FIG. 3C illustrates a diagram of the heart 300 after delivery of the PCI therapy and a reperfusion therapy, such as a therapy delivered by the intravascular reperfusion therapy device 160. In particular, FIG. 3C illustrates a diagram of the heart 300 following delivery of a reperfusion therapy targeting the first area of tissue 310. According to techniques described in greater detail below, reperfusion therapy may be delivered by the intravascular reperfusion therapy device 160 to reduce or minimize injury at and/or to improve blood flow to tissue where blood is re-perfusing (e.g., an area of tissue receiving an increase in blood flow), such as the first area of tissue 310. In particular, reperfusion therapy may affect a distribution of blood flow through the targeted tissue such that blood flow perfuses (e.g., distributes to) and/or increases throughout the tissue, including through areas of the tissue that are inflamed or have oxidative damage. Accordingly, delivery of the reperfusion therapy targeting an area of tissue may restore blood flow to a healthy amount or an amount exceeding the blood flow resulting from the PCI therapy alone. For instance, in the illustrated embodiment, the health and/or functioning of (e.g., the blood flow to) the first area of tissue 310 is shown as being fully restored by the reperfusion therapy, as indicated by the fill pattern of the first area of tissue 310 matching the second area of tissue 320. In some embodiments, however, the reperfusion therapy may restore the health and/or functioning of (e.g., the blood flow to) tissue to a level greater than a level resulting from the PCI but less than a level at an area of tissue, such as the second area of tissue 312, that was relatively unaffected by a blockage (e.g., associated with a different vessel than the vessel having the blockage). Particular mechanisms for controlling, using one or more components of the system 100, the reperfusion of an area of tissue associated with (e.g., configured to receive blood/oxygen from) a vessel that receives a PCI therapy (e.g., a vessel that has an occlusion) and/or otherwise receives an increase in blood flow are described herein.

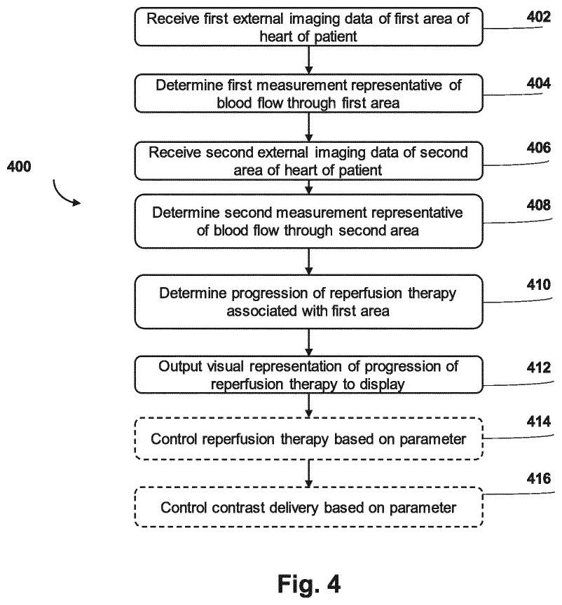

FIG. 4 is a flow diagram of a method 400 of evaluating (e.g., assessing), displaying, and/or controlling (e.g., modifying) a progress of reperfusion of a tissue, according to aspects of the present disclosure. In some embodiments, the method 400 may be used to control a reperfusion therapy, which may be delivered via a device, such as the intravascular reperfusion therapy device 160 of FIG. 1. As illustrated, the method 400 includes a number of enumerated steps, but embodiments of the method 400 may include additional steps before, after, or in between the enumerated steps. In some embodiments, one or more of the enumerated steps may be omitted, performed in a different order, or performed concurrently. The steps of the method 400 can be carried out by any suitable component within the system 100 and all steps need not be carried out by the same component. In some embodiments, one or more steps of the method 400 can be performed by, or at the direction of, a processor circuit of the system 100, including, the processing system 110 (e.g., the processor 210 (FIG. 2)) or any other component.

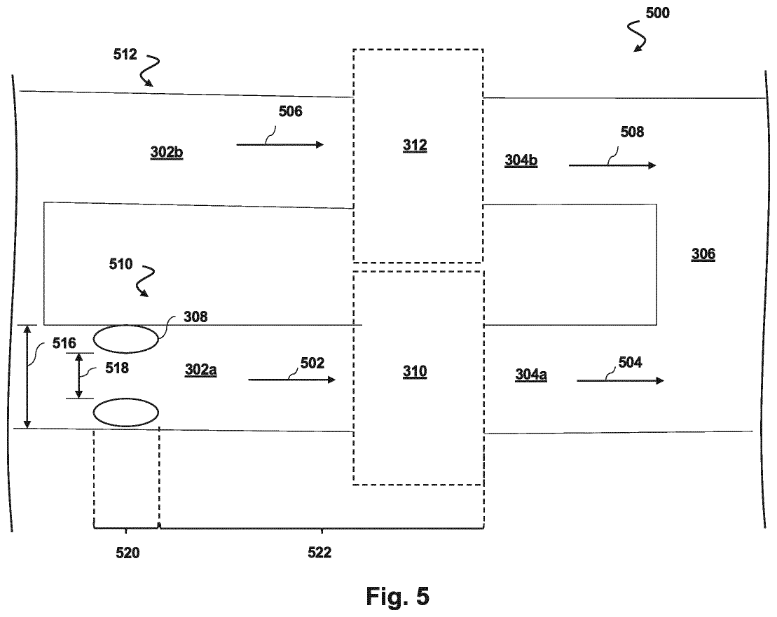

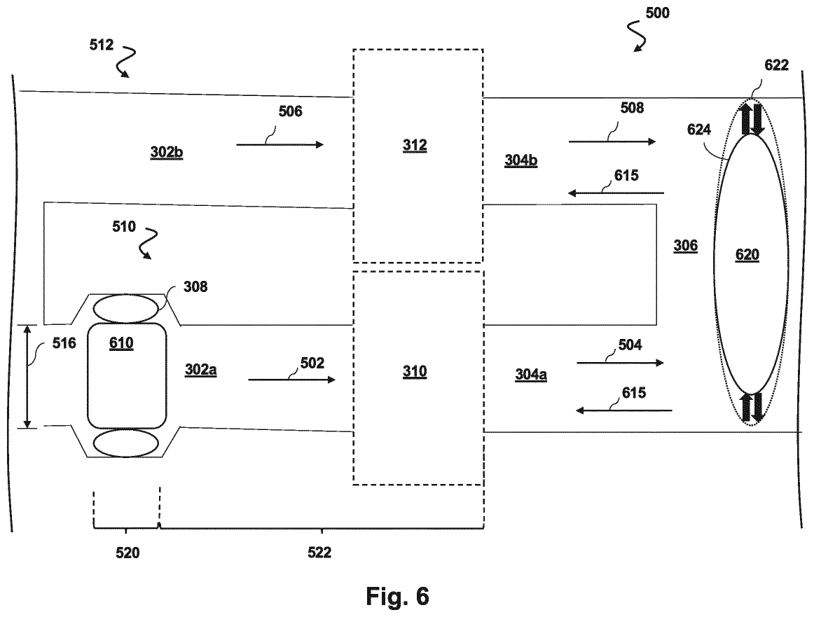

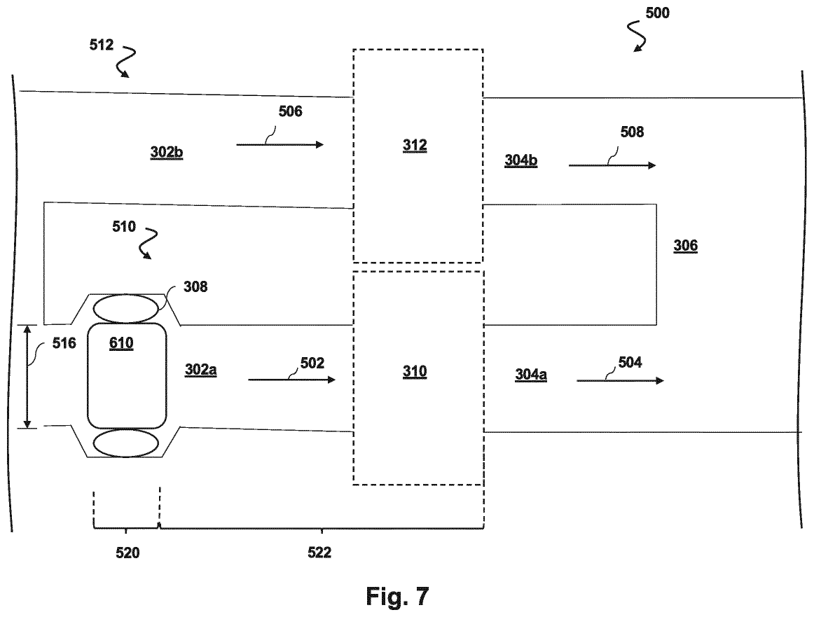

At step 402, the method 400 involves receiving first external imaging data of a first area of a heart of a patient. In particular, the first area of the heart may be vasculature and/or tissue associated with (e.g., receiving blood flow from) a first vessel with a blockage (e.g., a lesion), such as the first area 510 illustrated in FIGS. 5-7. In some embodiments, the first area of the heart may be vasculature and/or tissue associated with (e.g., receiving blood flow from) a first vessel with a previous blockage (as opposed to a current blockage). In some embodiments, the first area may be an area that experiences or has experienced a period of reduced blood flow/ischemia. As described in greater detail below, the first area 510 is depicted before a PCI therapy is administered in FIG. 5, after the PCI therapy is administered and/or while a reperfusion therapy is administered in FIG. 6, and after the reperfusion therapy is administered in FIG. 7. In some embodiments, the first external imaging data may include imaging data of the first area (e.g., first area 510) after the PCI therapy is administered. In this regard, the first external imaging data may correspond to imaging data of the first area 510 as depicted in FIG. 6. The first external imaging data may also include imaging data of the first area before the PCI therapy in administered, in some cases. In this regard, the first external imaging data may correspond to imaging data of the first are 510 as depicted in FIG. 5.

In some embodiments, receiving the first external imaging data may involve receiving the first external imaging data at the processing system 110. Further, the first external imaging data may include one or more external images, which may be obtained by the external imaging device 140. For instance, the first external imaging data may include one or more include x-ray images, CT images, MRI images, SPECT images, external ultrasound images, and/or the like. Accordingly, in some embodiments, the processing system 100 may receive the first external imaging data from the external imaging device 140 and/or a data storage device (e.g., memory device) in communication with the external imaging device 140. Further, the first external imaging data may include contrast (e.g., may be obtained using contrast), which may be provided intravenously to the patient via the contrast infusion pump 170. In this way, the appearance of the first area of the heart in the first external imaging data may depend on the contrast supplied to the patient, as well as the blood flow through the first area of the heart. For instance, as illustrated and described below with respect to FIGS. 5-7, the appearance of the first area 510 may vary from the appearance of other structures of the heart and/or anatomical features, and the appearance of the first area 510 may vary based on therapeutic procedures performed in association with the first area 510.

With reference now to FIG. 5, a schematic diagram of a portion 500 of a heart of a patient, which may correspond to a detailed view of a portion of the heart 300 illustrated in FIG. 3A, is illustrated. More specifically, FIG. 5 illustrates a first coronary artery 302a, which includes a blockage 308 (e.g., a stenosis) and is arranged to provide blood/oxygen to a first area of tissue 310, as indicated by the arrow 502. A first coronary vein 304a is arranged to carry deoxygenated blood away from the first area of tissue 310, as indicated by the arrow 504, and is illustrated as being in fluid communication with the coronary sinus 306. FIG. 5 further depicts a second coronary artery 302b, which lacks a blockage (e.g., is a healthy vessel) and is arranged to provide blood/oxygen to a second area of tissue 312, as indicated by arrow 506. A second coronary vein 304b is arranged to carry deoxygenated blood away from the second area of tissue 312, as indicated by arrow 508, and is illustrated as being in fluid communication with the coronary sinus 306. In some embodiments, the first area 510, which may include at least one of the first coronary artery 302a, the first area of tissue 310, or the first coronary vein 304a, may represent the first area of the heart included in the first external imaging data received at step 402 of the method 400 (FIG. 4). In that regard, the schematic diagram illustrated in FIG. 5 may be representative of one or more external images (e.g., an x-ray image, CT image, MRI image, and/or the like) of a portion of the heart. Further, the second area 512, which may include at least one of the second coronary artery 302b, the second area of tissue 312, or the second coronary vein 304b, may represent a second area of the heart included in second external imaging data received at step 406 of the method 400 (FIG. 4), as described in greater detail below. The imaging data and/or the imaging area can also include one, two, or more coronary arteries. The propagation of contrast through the one, two, or more coronary arteries can be an indication of the amount of relative perfusion of areas 310 and 312.

As described above with respect to FIGS. 3A-3B, the blockage 308 may disrupt blood flow through the first coronary artery 302a. In particular, the blockage 308 may reduce a diameter of a lumen of the first coronary 302a from a first diameter 516 to a smaller, second diameter 518 within the portion 520 of the vessel where the blockage 308 is located. This reduction in diameter may increase the resistance and/or the impedance to blood flowing through the portion 520, which may reduce blood/oxygen delivered distal of the blockage (e.g., within the distal area 522 including a portion of the coronary artery 302a and the first area of tissue 310). In this regard, the first area of tissue 310 (e.g., a portion of myocardium) may experience ischemia (e.g., a reduction in delivered blood/oxygen), which may damage the first area of tissue 310. In some embodiments, the first area 310 can be and/or include coronary artery 302a.

FIG. 5 further illustrates that the second coronary artery 302b is relatively healthy. That is, for example, the second coronary artery 302b lacks a stenosis (e.g., a blockage 308). As such, the flow of blood through the second coronary artery 302b may be uninterrupted by changes in the diameter of the lumen of the second coronary artery 302b. Accordingly, the second area of tissue 312 (e.g., a portion of myocardium) may receive a sufficient supply of blood/oxygen from the second coronary artery 302b and may remain relatively unaffected by the blockage 308. In some embodiments, the second area 312 can be and/or include coronary artery 302b.

In some embodiments, the difference in blood/oxygen delivered to the first area of tissue 310, which is associated with (e.g., receives blood flow from) a vessel having the blockage 308, and the second area of tissue 312, which is associated with (e.g., receives blood flow from) a different vessel may be detected using external imaging data. More specifically, external imaging data obtained using contrast may highlight differences in blood flow and/or blood delivery (e.g., perfusion) between different areas (e.g., blood vessels and/or tissues) of the heart. For instance, administration of a contrast agent into a patient's vasculature (e.g., via the contrast infusion pump 170) may change an appearance of the patient's blood and/or vasculature in an external image, which may facilitate contrast blush imaging and/or angiography, for example. As an illustrative example, if a patient is administered a contrast agent, such as an iodine-based compound, that absorbs external x-rays from an x-ray source (e.g., the external imaging device 140), features carrying the contrast agent appear darker (e.g., have a decreased exposure) in an x-ray image than features lacking the contrast agent. In this way, intravenously administering the contrast agent may result in an external image having relatively darker areas of vasculature and/or tissue that receive the contrast, which correspond to areas of vasculature and/or tissue that receive blood flow, in comparison with areas that do not receive the contrast (e.g., areas of vasculature and/or tissue that do not receive blood flow). In some embodiments, the contrast agent may include a contrast agent that may decrease absorption of the external x-rays from the x-ray source, such as carbon dioxide. In such cases, the features carrying the contrast agent (e.g., areas of vasculature and/or tissue that receive blood flow) may appear brighter in an external image than features lacking the contrast. As illustrated in FIG. 5, for example, the second area of tissue 312 is illustrated as having a first fill pattern, which is relatively brighter than the different, second fill pattern of the first area of tissue 310. The first fill pattern demonstrates that the second area of tissue 312 receives a first amount of blood flow, while the second fill pattern demonstrates that the first area of tissue 310 receives a second amount of blood flow that is relatively less than the first amount. Moreover, while certain contrast agents and their effect on x-ray imaging is described herein, embodiments are not limited thereto. In this regard, any suitable contrast agent may be used with any suitable external imaging to obtain external imaging data that differentiates between areas that receive blood flow and areas that do not receive blood flow.

FIG. 6 is a schematic diagram of the portion 500 of the heart following delivery of a PCI therapy 610. In some embodiments, FIG. 6 may correspond to a detailed view of a portion of the heart 300 illustrated in FIG. 3B. The PCI therapy 610 may be delivered by the intravascular lesion therapy device 150 and/or the PCI therapy 610 may be the intravascular lesion therapy device 150 itself. For instance, as described above with respect to FIG. 1, the PCI therapy 610 may involve ablation of the blockage 308, deployment of a balloon (e.g., angioplasty), stent, and/or drug, thrombectomy, atherectomy, and/or the like. In that regard, the PCI therapy 610 may involve a therapeutic procedure that widens the diameter available for blood flow in the portion 520 having the blockage 308 from the diameter 518. That is, for example, the PCI therapy 610 may involve widening the diameter of the lumen of the first coronary artery 302a, as shown. Additionally or alternatively, a size of the blockage 308 may be reduced to increase the diameter available for blood flow within the portion 520. Further, the PCI therapy 610 may involve the placement of a physical device, such as a stent, within the first coronary artery 302a. In some embodiments, the PCI therapy 610 may involve the use of a device (e.g., a guidewire or catheter) that is removed from the first coronary artery 302a when the PCI therapy 610 is completed. In that regard, the illustrated representation of the PCI therapy 610 positioned within the first coronary artery 302a is intended to be exemplary and not limiting.

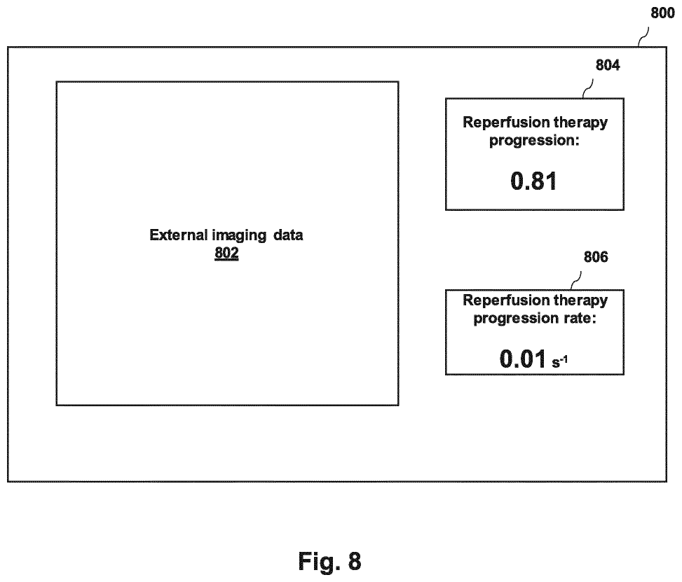

Turning back now to FIG. 4, the first external imaging data of the first area of the heart (e.g., received at step 402) may include external imaging data of the first area of the heart following delivery of a PCI therapy (e.g., PCI therapy 610), as described herein. Further, in some embodiments, the first area of the heart may be the only area within the first external imaging data. In some embodiments, for example, the processing system 110 may receive external imaging data from the external imaging device 140 that is taken in response to a user input (e.g., via the input device 130) to capture imaging data with respect to the first area. For instance, the processing system 110 may instruct and/or control the external imaging device 140 to obtain the first external imaging data such that it is specific to the first area. Additionally or alternatively, the first area may be identified within the first external imaging data. For instance, the first external imaging data may be displayed (e.g., at display 120) so that a user may provide a user input (e.g., via the input device 130) selecting the first area within the first external imaging data. To that end, the user may interact with a screen display (e.g., a graphical user interface (GUI)), such as the screen display illustrated in FIG. 8. Further, in some embodiments, the processing system 110 may automatically identify the first area. For instance, the processing system 110 may identify the first area based on the features of and/or included in the first external imaging data, such as features included in the first area 510 illustrated in FIG. 6. That is, for example, the processing system 110 may identify the first area based on the first area including a PCI therapy (e.g., a stent), a blockage, which may appear as a narrowing of a blood vessel within the external imaging data, and/or the like. In some embodiments, the features of and/or included in the first external imaging data can include blood flow (e.g., reduced blood flow). Additionally or alternatively, the processing system 110 may identify the first area based on the first area being associated with the intravascular reperfusion therapy device 160, which may be positioned proximate the first area, for example.

At step 404, the method 400 may involve determining a first measurement representative of blood flow through the first area (e.g., the identified and/or selected first area). More specifically, the method 400 may involve determining the first measurement based on the first external imaging data. For example, the processing system 110 may receive the first external imaging data from the external imaging device 140 (e.g., at step 402) and may determine the first measurement based on the received external imaging data. In some embodiments, the measurement may be a measurement of a parameter, such as a pixel value (e.g., a color and/or grayscale value), brightness, intensity, contrast, and/or the like, associated with the first area within the first external imaging. As described above, these parameters may depend on the flow of contrast through the first area, which indicates the flow of blood through the first area. As an illustrative example, a measurement of the intensity of the first area may increase for increasing levels of contrast flowing through the first area, which corresponds to increased blood flow through the first area. Continuing with this example, the measurement of the intensity of the first area may decrease with decreasing levels of contrast flowing through the first area, which corresponds to decreased blood flow through the first area. In some embodiments, the relationship between a measured parameter may depend on the type of contrast used. For instance, the color of the first area may depend on whether the contrast agent increases or decreases x-ray absorption in the first external imaging data. Further, the first measurement may be a wash-in rate of the contrast agent within the first area, a wash-out rate of the contrast agent from the first area, and/or the like. In this regard, the first measurement may be made based on one or more external images of the first external imaging data. Moreover, the measurement may be a peak value (e.g., a maximum value), a minimum value, an average value for a set time or across a certain number of images in the first imaging data, a median value, an integral, a derivative, and/or the like associated with a parameter corresponding to the first area.

In some embodiments, the first measurement may be determined with respect to an area of tissue included within the first area, such as the first area of tissue 310, which is included in the first area 510 illustrated in FIGS. 5-7. For instance, the first measurement may be determined based on contrast blush imaging techniques with respect to the first area of tissue. Additionally or alternatively, the first measurement may be determined with respect to a blood vessel included in the first area, such as the first coronary artery 302a. In this regard, the first measurement may be a measurement of contrast velocity through the blood vessel. Again, the measurement may be a peak measurement of the contrast velocity, an average measurement of the contrast velocity, an integral, a derivative, and/or the like.

At step 406, the method 400 may involve receiving second external imaging data of a second area of the heart of the patient. The second area of the heart included in the second external imaging data may be associated with a second blood vessel. In some embodiments, the second blood vessel may be different than the first blood vessel associated with the first area of the heart (e.g., included in the first external imaging data). Moreover, the second blood vessel may lack a blockage and/or may be arranged to provide tissue with a relatively healthier amount of blood/oxygen than the first blood vessel. In this regard, the second area 512 depicted in FIGS. 5-7 may be representative of the second area of the heart. To that end, the second area 512 is shown as being associated with the second coronary artery 302b and may include the second coronary artery 302b, the second area of tissue 312, and/or the second coronary vein 304b.

As described with respect to the first external imaging data, receiving the second external imaging data may involve receiving the second external imaging data at the processing system 110. Further, the second external imaging data may include one or more external images of the second area obtained by the external imaging device 140. For instance, the second external imaging data may include one or more include x-ray images, CT images, MRI images, SPECT images, external ultrasound images, and/or the like. Accordingly, in some embodiments, the processing system 100 may receive the second external imaging data from the external imaging device 140 and/or a data storage device (e.g., memory device) in communication with the external imaging device 140. Further, the second external imaging data may include contrast (e.g., may be obtained using contrast), which may be provided to the patient via the contrast infusion pump 170. In this way, the appearance of the second area of the heart in the second external imaging data may depend on the contrast supplied to the patient, as well as the blood flow through the second area of the heart. Further, in some embodiments, the second external imaging data may be obtained with the same contrast agent or a different contrast agent than the first external imaging data.

In some embodiments, receiving the second external imaging data may occur concurrently with receiving the first external imaging data (e.g., at step 402). For instance, the processing system 110 may receive external imaging data that includes the first external imaging data and the second external imaging data. For instance, a first portion of the external imaging data may correspond to the first external imaging data and a second portion of the external imaging data may correspond to the second external imaging data. For instance, a first subset of images within the external imaging data may correspond to the first external imaging data of the first area, and a second subset of images within the external imaging data may correspond to the second external imaging data of the second area. Additionally or alternatively, the first external imaging data of the first area may correspond to a first region (e.g., area of pixels) within the images of the external imaging data, while the second external imaging data of the second area may correspond to a second region within the images of the external imaging data.

In some embodiments, the second area of the heart may be the only area within the second external imaging data. In some embodiments, for example, the processing system 110 may receive external imaging data from the external imaging device 140 that is taken in response to a user input (e.g., via the input device 130) to capture imaging data with respect to the first area. For instance, the processing system 110 may instruct and/or control the external imaging device 140 to obtain the second external imaging data such that it is specific to the second area. Additionally or alternatively, the second area may be identified within the second external imaging data and/or external imaging data including both the first and second external imaging data. For instance, the second external imaging data may be displayed (e.g., at display 120) so that a user may provide a user input (e.g., via the input device 130) selecting the second area within the second external imaging data. To that end, the user may interact with a screen display (e.g., a graphical user interface (GUI)), such as the screen display illustrated in FIG. 8. Further, in some embodiments, the processing system 110 may automatically identify the second area. For instance, the processing system 110 may identify the second area based on the features of included in the second external imaging data, such as features included in the second area 512 illustrated in FIG. 6. That is, for example, the processing system 110 may identify the second area based on the second area including being associated with a second vessel, such as a vessel that lacks a blockage.

At step 408, the method 400 may involve determining a second measurement representative of blood flow through the second area. More specifically, the method 400 may involve determining the second measurement based on the second external imaging data. For example, the processing system 110 may receive the second external imaging data from the external imaging device 140 (e.g., at step 406) and may determine the second measurement based on the received external imaging data. The determination of the second measurement based on the second external imaging data may be substantially similar to the determination of the measurement of any combination of the various parameters described with respect to the first measurement and the first external imaging data. Accordingly, for the sake of brevity, the details of the determination of the second measurement will not be repeated.

In some embodiments, the second measurement may correspond to a value of the same parameter or combination of parameters measured (e.g., determined) for the first measurement. For instance, each of the first measurement and the second measurement may correspond to a measurement of intensity, contrast velocity, wash-in rate, wash-out rate, and/or the like for the respective first or second area of the heart. Additionally or alternatively, the second measurement may correspond to a value of a different parameter than the first measurement. As an illustrative example, a first contrast agent may be used in the first external imaging data, and a different, second contrast agent may be used in the second external imaging data that affects an appearance of features differently than the first contrast agent. Accordingly, the second measurement may be made with respect to the effect of the second contrast agent, which may involve measurement of the inverse of a parameter used in the first measurement or measurement of a parameter with respect to a normalization (e.g., calibration), for example. In any case, the first measurement may be representative of blood flowing through an area of the heart experiencing reperfusion (e.g., that is receiving an increase in blood flow following an event, such as PCI therapy), and the second measurement may be representative of blood flowing through an area of the heart that has remained relatively unaffected by a blockage or reperfusion (e.g., blood flowing through a relatively healthy area).

At step 410, the method 400 may involve determining a progression of a reperfusion therapy associated with the first area. In some embodiments, the reperfusion therapy may correspond to a reperfusion therapy delivered to and/or in association with the first area. For instance, the reperfusion therapy may be a therapy delivered by the intravascular reperfusion therapy device 160. Moreover, the reperfusion therapy may regulate blood flow to the target tissue in some manner, as illustrated in FIG. 6.

With reference now to FIG. 6, a reperfusion therapy 620 is illustrated as being delivered in association with the first area of tissue 310. In particular, the reperfusion therapy 620 is shown as being delivered at a venous side of the first tissue 310 (e.g., within the coronary sinus 306). In some embodiments, reperfusion therapy delivered to a venous side of the first area of tissue 310 may involve obstructing vasculature on the venous side of the first area of tissue 310. For instance, the intravascular reperfusion therapy device 160 may include a balloon configured to selectively expand (e.g., under the control of the processing system 110) to deliver the reperfusion therapy 620. More specifically, the balloon may oscillate between a first configuration 622, which may fully obstruct the vasculature, and a second configuration 624, which may partially obstruct the vasculature. By obstructing the vasculature in this manner, the intravascular reperfusion therapy device 160 may increase pressure on the venous side of the tissue, which may encourage better (e.g., increased and/or more even) distribution of blood flow through the first area of tissue 310, especially the damaged or inflamed portions of the area of tissue 310. In particular, the balloon may generate a back pressure and/or back flow in a direction indicated by arrow 615 via restriction of blood flow through the coronary vein in the opposite direction (e.g., indicated by arrow 504 and by arrow 508). The reperfusion therapy 620 may additionally or alternatively involve the supply of fluids (e.g., blood and/or saline) to affect a pressure change (e.g., introduce a back pressure at the venous side of the first tissue 310) within the vasculature, temperature control (e.g., supply of cooling temperatures to the tissue, which may reduce inflammation within the first area of tissue 310, and/or the like. Moreover, while the reperfusion therapy 620 is illustrated as being supplied within the venous vasculature (e.g., the coronary sinus 306), reperfusion therapy may additionally or alternatively be supplied to arterial vasculature, such as within the first coronary artery 302a. In cases where the reperfusion therapy is delivered arterially, the intravascular reperfusion therapy device 160 may be configured to obstruct arterial vasculature (e.g., the first coronary artery 302a) at varying degrees to control the flow of blood into the first area of tissue 310. As an illustrative example, the reperfusion therapy device 160 may include a balloon that obstructs the first coronary artery 302a to a first degree (e.g., fully obstructs), and as the balloon deflates to a second degree of obstruction, increased blood flow may be provided to the first area of tissue 310. In this way, blood flow to the tissue 310 may be increased at a control rate following the PCI therapy 610. Reperfusion on the arterial side may additionally or alternatively involve the delivery of fluids, temperature control, and/or the like.

While the present disclosure describes embodiments of the intravascular reperfusion therapy device including a balloon, it is understood that the intravascular reperfusion therapy device can include any suitable structure that selectively restricts blood flow and/or generates back pressure. For example, the structure can be the balloon. In some embodiments, the structure can be a controllable pump and a lumen for fluid (e.g., saline, oxygen). The structure can be an obstruction in some embodiments. For example, the obstruction can be an expandable structure (e.g., open/close valve or valve stent, expandable basket/multi-arm structure with or without material in between the arms) that can be controlled to be in one state (closed valve or expanded basket/arms restricting blood flow) and another state (open valve or contracted basket/arms allowing blood flow).

Turning back now to FIG. 4, determining a progression of the reperfusion therapy (e.g., at step 410) may involve determining a characteristic of blood flow through the first area of the heart. That is, for example, step 410 of the method 400 may involve determining whether tissue associated with a vessel with a blockage is receiving a healthy amount of blood flow. As described above, the first measurement may be representative of the blood flow through the first area, and the second measurement may be representative of the blood flow through the second area (e.g., a relatively healthy area). Accordingly, the second measurement may provide a benchmark and/or a point of reference to determine the health of the first area (e.g., using the first measurement). To that end, a comparison of the first measurement and the second measurement may provide an indication of the relative health (e.g., relative blood flow) of the first area with respect to the second area of the heart in terms of blood flow. In this regard, determining the progression of the reperfusion therapy may involve determining a parameter representative of blood flow with respect to the first area and the second area based on the first measurement and the second measurement.

In some embodiments, the processing system 110 may determine the progression of the reperfusion therapy. Accordingly, the processing system 110 may determine the parameter representative of the blood flow with respect to the first and second areas based on the first and second measurements. In this regard, the parameter may be representative of a relative blood flow (e.g., relative blood perfusion) between the first area and the second area. In particular, the processing system 110 may determine the parameter based on a comparison of the first and second measurements. To that end, the parameter may provide an indication of a relative blood perfusion through the first area in comparison with the second area. In some embodiments, for example, the processing system 110 may determine a ratio of the first and second measurements. In some embodiments, the processing system 110 may further determine the progression of the reperfusion therapy by determining an integral or a derivative of the parameter with respect to time. For instance, by determining the derivative of the parameter, the processing system 110 may determine a rate of change of the relationship between the first and second measurements over time. In this way, the processing system 110 may determine whether the reperfusion therapy is improving blood flow to the first area (e.g., whether the blood flow to the first area is becoming more similar to the blood flow to the second area) and/or the rate at which the reperfusion therapy is impacting the blood flow to the first area. That is, for example, the processing system 110 may determine an efficacy and/or an efficiency of the reperfusion therapy.

In some embodiments, the processing system 110 may further relate the determined parameter and/or derivative to the progression of the reperfusion therapy. For instance, the processing system 110 may compare the determined parameter and/or the derivative with one or more thresholds to relate the determined parameter to the progression of the reperfusion therapy. As an illustrative example, a first range of parameters and/or derivatives may correspond to the reperfusion therapy demonstrating improvement for the first area, a second range may correspond to the reperfusion therapy demonstrating worsening blood flow conditions at the first area, a third range may correspond to the reperfusion therapy demonstrating having no effect or a relatively small effect on the first area, and/or the like. Such thresholds may additionally or alternatively be used to quantify a degree of an effect the reperfusion therapy has on the first area. As an additional example of thresholds, a first range of parameters and/or derivatives may correspond to blood flow within the first area being healthy, while a second range may correspond to blood flow within the first area being not healthy. Moreover, the thresholds may be used to determine when the reperfusion therapy is complete, such as when the blood flow through the first area has reached the healthy, first range, plateaued at a maximum value, the blood flow through the first area approaches the blood flow through the second area, and/or the like. In some embodiments, the processing system 110 may additionally or alternatively compare the determined parameter and/or derivative to one or more previous respective values to relate the progress of the reperfusion therapy. In that regard, the processing system 110 may determine the change in blood flow within the first area over time to determine the progression of the reperfusion therapy.

At step 412, the method 400 may involve outputting a visual representation of the progression of the reperfusion therapy to a display. For instance, the processing system 110 may output a screen display including a representation of the progression of the reperfusion therapy to the display device 120. An example of a screen display including a representation of the progression of the reperfusion therapy is illustrated and described with respect to FIG. 8.

FIG. 8 illustrates a screen display 800, which includes a graphical representation of external imaging data 802, as well as visual representations of the progress of the reperfusion therapy. The external imaging data 802 may be obtained by the external imaging device 140 and may be output to the display 120 by the processing system 110. Further, the external imaging data 802 may correspond to the first external imaging data and/or the second external imaging data (e.g., received at step 402 or 406, respectively). In that regard, the external imaging data 802 may depict the first area 510 and the second area 512, and the external imaging data 802 may include contrast. Accordingly, the external imaging data 802 may facilitate a visual comparison between the blood flow through first area 510 and the second area 512, as generally illustrated by the differences in fill patterns shown in the first tissue area 310 and the second tissue area 312 in FIGS. 5-7. Further, the external imaging data 802 may include one or more static images or an image stream. In some embodiments, navigating through the static images (e.g., using the input device 130) or watching a progression of the image stream may provide an indication of the progress of the reperfusion therapy, as the visual appearance of contrast delivered to the first area will change based on an impact of the therapy.

The progression of the reperfusion therapy may further be provided via a visual representation associated with the parameter 804 indicative of a comparison of the first and second measurements (e.g., the parameter determined at step 410) and/or a visual representation associated with the derivative of the parameter 806. In some embodiments, the visual representation associated with the parameter 804 and/or the visual representation associated with the derivative of the parameter 806 may include a numerical representation, a graph, chart, or plot, a textual representation, one or more symbols, and/or the like. Further, in some embodiments, the parameter and/or the derivative of the parameter may be compared to one or more thresholds. Accordingly, the visual representation associated with the parameter 804 and/or the visual representation associated with the derivative of the parameter 806 may indicate the relationship of the parameter and/or the derivative of the parameter with respective one or more thresholds. For instance, based on a comparison of the parameter or the derivative with a respective threshold, the progression of the reperfusion therapy may be represented textually as “improving,” “worsening,” “no change,” “complete,” and/or the like. The progression of the reperfusion therapy may additionally or alternatively be indicated with respective symbols, colors, and/or the like associated with the statuses “improving,” “worsening,” “no change,” “complete,” and/or the like.

With reference again to FIG. 4, at step 414, the method 400 may involve controlling a reperfusion therapy (e.g., the reperfusion therapy 620) based on the parameter (e.g., based on the progression of the reperfusion therapy). In some embodiments, for example, the processing system 110 may control (e.g., instruct) the intravascular reperfusion therapy device 160 based on the progression of the reperfusion therapy (e.g., based on the parameter). For instance, in the case of reperfusion therapy 620 being delivered venously, the processing system 110 may adjust a frequency, duty cycle, and/or the like of the oscillation of a balloon of the intravascular reperfusion therapy device 160 between the first configuration 622 and the second configuration 624 (FIG. 6). In the case of reperfusion therapy 620 being delivered arterially, the processing system 110 may adjust a degree of obstruction (e.g., inflation) within the vasculature caused by the intravascular reperfusion therapy device 160. The processing system 110 may additionally or alternatively adjust temperature control, fluid delivery, and/or the like provided at the intravascular reperfusion therapy device 160. In particular, based on the progression of the reperfusion therapy indicating the progression is relatively poor, the processing system 110 may instruct the intravascular reperfusion therapy device 160 to increase back pressure resulting on the venous side of the tissue (e.g., increase venous obstruction), decrease the temperature, decrease arterial obstruction (e.g., increase arterial blood flow), and/or otherwise increase blood flow through the first area 310. For instance, the processing system 110 may control the device 160 to increase the frequency and/or period that the device 160 is in the first configuration 622. Further, based on the progression of the reperfusion therapy indicating the progression is relatively good, the processing system 110 may instruct the intravascular reperfusion therapy device 160 to maintain current therapy and/or to gradually terminate (e.g., ease off) delivery of the therapy. In general, the processing system 110 may adjust one or more characteristics of the intravascular reperfusion therapy device 160 operation based on a feedback loop with the intravascular reperfusion therapy device and the determined progression of the reperfusion therapy. In that regard, embodiments are not limited to the mechanisms of controlling the intravascular reperfusion therapy device described herein. Moreover, it may be appreciated that steps of the method 400 may be repeated, such that the processing system 110 may continually determine a current progression of the reperfusion therapy (e.g., based on updated first and second measurements) and adaptively adjust the operation of the intravascular reperfusion therapy device 160.

In some embodiments, responsive to determining that the blood flow through the first area has reached a healthy level (e.g., satisfies a threshold), has reached a level substantially similar to blood flow through the second area, has plateaued at a maximum value, the processing system 110 may determine that the progression of the reperfusion therapy has reached completion. In such cases, the processing system 110 may control the intravascular reperfusion therapy device 160 to terminate delivery of the reperfusion therapy. FIG. 7 provides an illustrative example of the portion of the heart 500 after reperfusion therapy 620 is complete, as illustrated by the absence of the intravascular reperfusion therapy device 160 and/or the reperfusion therapy 620. In that regard, FIG. 7 may correspond to a detailed view of a portion of the heart 300 illustrated in FIG. 3C. As described above, when reperfusion therapy is complete, the blood flow in the first area of tissue 310 may be greater than a blood flow amount following a PCI therapy and may be substantially similar to the blood flow in the second area of tissue 312 (as illustrated by the matching fill patterns of 310 and 312).

In some embodiments, the method 400 may optionally include the step 414 (as illustrated by the dashed lines). In that regard, the reperfusion therapy may additionally or alternatively be controlled based on one or more user inputs, which may be received via the input device 130, for example. To that end, a user may provide an input at the input device 130, and the processing system 110 may control operation of the intravascular reperfusion therapy device 160 based on the input.

At step 416, the method 400 may involve controlling contrast delivery (e.g., delivery of a contrast agent) based on the parameter, the first external imaging data, and/or the second external imaging data. For instance, based on determining blood flow through the first area, the processing system 110 may adjust the delivery of contrast agent to the first area. In particular, the processing system 110 may reduce a delivery rate and/or an amount of delivered contrast agent responsive to determining that the contrast agent flows relatively slowly through the first area such that a first dose (e.g., bolus) of contrast may completely clear from the first area before a subsequent dose is delivered to the first area. In some cases, the processing system 110 may increase a delivery rate of the contrast agent and/or an amount of delivered contrast agent so that sufficient levels of contrast agent may simultaneously be supplied across the first area to facilitate imaging of the entire first area, for example. Further in some embodiments, the processing system 110 may coordinate control of the contrast infusion pump 170 and the external imaging device 140 such that the external imaging device 140 may obtain the first external imaging data and/or the second external imaging data in a manner (e.g., at a frequency, duration, and/or the like) that the processing system 110 may utilize to determine the first and second measurement based on characteristics of the contrast within the imaging data.

In some embodiments, the processing system 110 may additionally or alternatively control the contrast infusion pump to selectively deliver a first contrast agent or a second contrast agent to the patient. In this manner, the processing system 110 may reduce stress on the anatomy of the patient that may result from prolonged delivery of a particular contrast agent. In some embodiments, the processing system 110 may adjust (e.g., normalize and/or calibrate) the first measurement (at step 404) and/or the second measurement (at step 408) based on the contrast agent delivered by the contrast infusion pump 170 in the first external imaging data and/or the second external imaging data, respectively. To that end, the determination of the progression of the reperfusion therapy may not be affected by changes in the type of contrast agent delivered to the patient.

In some embodiments, the method 400 may optionally include the step 416 (as illustrated by the dashed lines). In that regard, the contrast infusion pump 170 may additionally or alternatively be controlled based on one or more user inputs, which may be received via the input device 130, for example. To that end, a user may provide an input at the input device 130, and the processing system 110 may control operation of the contrast infusion pump 170 based on the input. Additionally or alternatively, the contrast infusion pump 170 may be configured to run an automated program (e.g., schedule) of contrast agent delivery.

While the system 100 and the method 400 are described herein as being employed for evaluating (e.g., assessing) and/or controlling reperfusion therapy, embodiments are not limited thereto. In that regard, the techniques described herein may additionally or alternatively be applied to microvascular disease (e.g., affecting capillary beds, for example) in any portion of a patient's anatomy (e.g., within or separate from the heart) and/or to nonobstructive coronary artery disease. Moreover, assessment and/or control of blood flow through particular tissue and/or capillary beds may be performed with or without a PCI therapy being performed on a vessel associated with the tissue and/or capillary beds. Further, in addition to or alternative of receiving second external imaging data of a second area and determining a measurement representative of blood flow through the second area, external imaging data of another patient or external imaging data taken of the first area of the heart of the patient at a different time than the first external imaging data (e.g., an earlier image of the first area) may be used to determine the progression of the reperfusion therapy for the first area. In that regard, determining the progression of the reperfusion therapy may be performed the first external imaging data and any suitable baseline of healthy blood flow through tissue.

A person of ordinary skill in the art will recognize that the present disclosure advantageously provides a system and method suitable to evaluate (e.g., assess) and/or control (e.g., adjust) a reperfusion therapy associated with an area of tissue. In particular, the techniques described herein provide an indication of a progression of the reperfusion therapy for first tissue associated with a blockage (e.g., a stenosis) based on a comparison of second tissue (e.g., relatively healthy tissue) with the first tissue. The logical operations making up the embodiments of the technology described herein are referred to variously as operations, steps, objects, elements, components, regions, etc. Furthermore, it should be understood that these may occur in any order, unless explicitly claimed otherwise or a specific order is inherently necessitated by the claim language.

It should further be understood that the described technology may be employed in a variety of different applications, including but not limited to human medicine, veterinary medicine, education and inspection. All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the intraluminal imaging system. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A system, comprising:
a processor circuit configured to:
receive first external imaging data of a first area of a heart of a patient associated with a first blood vessel with a blockage, wherein the first external imaging data includes blood flow through the first area;
determine a first measurement representative of the blood flow through the first area;
receive second external imaging data of a different, second area of the heart associated with a different, second blood vessel of the heart lacking the blockage, wherein the second external imaging data includes blood flow through the second area;
determine a second measurement representative of the blood flow through the second area;
determine a progression of a reperfusion therapy associated with the first area; and
output a visual representation of the progression of the reperfusion therapy to a display in communication with the processor circuit,
wherein, to determine the progression of the reperfusion therapy, the processor circuit is configured to determine a parameter representative of a relative blood flow between the first area and the second area based on the first measurement and the second measurement.

2. The system of claim 1, wherein the first external imaging data and the second external imaging data are obtained with contrast agent.

3. The system of claim 2, wherein the processor circuit is further configured to:
control, based on the parameter, an infusion pump in communication with the processor circuit to deliver the contrast agent to the first area and the second area.

4. The system of claim 3, further comprising the infusion pump.

5. The system of claim 1, wherein the visual representation of the progression of the reperfusion therapy comprises a visual representation of a derivative with respect to time of the parameter.

6. The system of claim 1, wherein, to determine the parameter, the processor circuit is configured to:
determine a ratio of the first measurement and the second measurement.

7. The system of claim 1, wherein the first measurement comprises at least one of a wash-in rate, a wash-out rate, an intensity of the first external imaging data, a brightness of the first external imaging data, or a contrast velocity.

8. The system of claim 1, wherein the processor circuit is further configured to:
receive selection of the first area,
wherein the processor circuit is further configured to determine the first measurement responsive to the selection of the first area.

9. The system of claim 1, wherein the processor circuit is further configured to:
identify the first area based on one or more features of the first external imaging data,
wherein the processor circuit is further configured to determine the first measurement responsive to the identification of the first area.

10. The system of claim 9, wherein the one or more features of the first external imaging data comprise a stent, intravascular reperfusion therapy device, or the blockage.

11. The system of claim 1, wherein the first external imaging data comprises an x-ray image of the first area.

12. The system of claim 1, wherein the first area comprises a first portion of a myocardium of the heart and the second area comprises a different, second portion of the myocardium.

13. The system of claim 1, wherein the first blood vessel comprises a first coronary artery and the second blood vessel comprises a second coronary artery.

14. The system of claim 13, wherein the processor circuit is further configured to:
control delivery of the reperfusion therapy based on the parameter.

15. The system of claim 14, wherein, to control the delivery of the reperfusion therapy, the processor circuit is configured to:
instruct an intravascular reperfusion therapy device in communication with the processor circuit and positioned within a vessel of the patient to control the blood flow through the first area.

16. The system of claim 15, wherein the vessel comprises a coronary vein.

17. The system of claim 15, further comprising the intravascular reperfusion therapy device.

18. A system, comprising:
a processor circuit configured to:
receive first x-ray imaging data of a first area of a heart of a patient, wherein the first x-ray imaging data comprises blood flow through the first area from a first blood vessel with a blockage, wherein the first area of the heart comprises at least one of a first portion of the myocardium or the first blood vessel, wherein the first blood vessel comprises a first coronary artery;
determine, using the first x-ray imaging data, a first measurement representative of the blood flow through the first area based on a contrast agent within the first area;
receive second x-ray imaging data of a different, second area of the heart, wherein the second x-ray imaging data comprises blood flow through the second area from a different, second blood vessel lacking the blockage, wherein the second area comprises at least one of a second portion of the myocardium or the second blood vessel, wherein the second blood vessel comprises a second coronary artery;
determine, using the second x-ray imaging data, a second measurement representative of the blood flow through the second area based on the contrast agent within the second area;
determine a progression of a reperfusion therapy associated with the first area; and
output a visual representation of the progression of the reperfusion therapy to a display in communication with the processor circuit,
wherein, to determine the progression of the reperfusion therapy, the processor circuit is configured to determine a parameter representative of a relative blood flow between the first area and the second area based on the first measurement and the second measurement.
